(19) United States
(12) Patent Application Publication
(10) Pub. No.: US 2008/0177266 A1
(43) Pub. Date: Jul. 24, 2008

(54) Adjustable height rasp

(75) Inventors: Newton H. Metcalf, Memphis, TN (US); Richard J. Thiele, Collierville, TN (US)

Correspondence Address: Medtronic;Spinal and Biologics, 2600 Sofamor Danek Drive, Memphis, TN, 38132, US

(73) Assignee: Warsaw Orthopedic, Inc.

(21) Appl. No.: 11/583,659

(22) Filed: Oct. 18, 2006

(51) Int. Cl. A61B 17/00

(52) U.S. Cl. 606/80

(57) ABSTRACT

Embodiments of an adjustable rasping tool for use in orthopedic medical procedures are disclosed. In certain embodiments, a body having two arms extending from a base and a tip portion on each arm is provided, with a shaft between the arms connected to a handle that is rotatably coupled to the base of the body. The shaft has an extended distal portion that contacts the tip portions of the arms. As the shaft is moved proximally (e.g. by rotating the handle in a given direction), the extended distal portion moves with respect to the tip portions and forces them apart. Methods for using embodiments of the disclosed structure are also described.

The present disclosure relates to tools useful in orthopedic surgery, and in particular to tools for preparing surfaces of one or more vertebrae or other bones or tissues.

In the field of orthopedic surgery, it is well-known to implant apparatus into or between bones and/or other tissue in order to provide support, correction, or other therapeutic benefit. For example, in the field of spinal orthopedic surgery, devices may be placed in an intervertebral space (e.g. between adjacent vertebrae) to replace diseased, injured or malformed tissue that has been removed. Such devices can include fusion promoting devices such as cages or intervertebral disk prostheses for maintaining spacing between vertebrae. These and other intervertebral devices contact vertebrae and/or adjacent tissue, commonly two facing vertebral endplates.

Insertion of such devices generally involves removal of disk tissue, i.e. a partial or complete diskectomy, to provide a space for the device. Once an appropriate space for the device is identified and cleared (if necessary), commonly the tissue that is going to contact the intervertebral device is prepared for the device. In many cases, such preparation can take the form of flattening, shaping, roughening or otherwise configuring a vertebral endplate or other tissue. In this way, the intervertebral device or associated structure or matter can fit more closely to or obtain greater purchase with the tissue it contacts. Following such preparation, the device can be inserted between the vertebrae. Frequently, some distraction of the vertebrae is necessary, spreading the vertebrae sufficiently to allow the device to be inserted between them. Release of the distraction allows the vertebrae or associated tissue to settle against the device.

Commonly the surgeon must move the tissues (e.g. vertebrae) apart with a separate tool or set of tools in order to maneuver the orthopedic site preparation tools into the proper place. For example, a first distraction tool for spreading vertebrae must be inserted in an intervertebral space, followed by a preparation tool maneuvered with respect to the distraction tool to the desired place by the surgeon. Such maneuvering can be quite difficult, particularly in small spaces between vertebrae. Further, if the distraction by the distraction tool is not exact, a preparation tool will have space or “play” that can make the preparation less exact or more difficult. Even if the distraction is exact, if material is removed by the preparation tool a similar “play” is created.

### SUMMARY

Among other things, there are disclosed embodiments tool for use in spinal orthopedic surgery, which may include a handle having a longitudinal axis, a body having a base connected to the handle and a plurality of arms each having a tip portion, each such tip portion having a relatively internal sloped surface and a relatively external surface, at least part of at least one of the external surfaces being a rasping surface, and the arms have an unstressed position relative to each other. Further, the tool may include a shaft having a proximal portion operatively connected to the handle and a distal portion having an expanded part that contacts the relatively internal sloped portions of the tip portions, whereby operating the handle in a first mode moves the expanded part against the relatively internal sloped portions to force the arms apart, and operating the handle in a second mode moves the expanded part relative to the relatively internal sloped portions to allow the arms to move toward the unstressed relative position.

In certain embodiments, the handle may be rotatable with respect to the body and threadedly connected to the shaft, and the first mode can include turning the handle relative to the body in a first direction, and the second mode can include turning the handle relative to the body in a second direction substantially opposite to the first direction. The body can include only two arms, and in the unstressed relative position of the arms, the arms define a slot between them. The shaft can include a boss that extends through at least part of the slot and to either side of the shaft and substantially perpendicularly to a longitudinal axis of the shaft. The shaft may extend within the slot and through the base. The extended part of said shaft can be tapered, e.g. forming at least part of a cone. At least one of the tip portions can include at least one hole from the relatively exterior surface to the relatively interior surface, and such hole(s) may be substantially perpendicular to a shaft longitudinal axis. At least one of said tip portions may include a relatively distal surface adjacent the relatively internal surface, said distal surface being substantially perpendicular to a shaft longitudinal axis. The arms can taper from the base toward the tip portions, and the tip portions can have a width greater than the portion of the arms adjacent them.

An embodiment of an orthopedic surgical apparatus disclosed herein may include a body having a base, first and second longitudinal arms extending from said defining a slot between them, and a longitudinal axis along said slot. The base has a hole therethrough substantially along the axis, and the first arm has an internal surface and a first tip portion, the first tip portion having an external rasping surface and an internal sloped surface. The second arm has an internal surface facing the internal surface of the first arm and a second tip portion facing the first tip portion, the second tip portion having an external surface and an internal sloped surface. The first and second arms have an unstressed relative position such that the internal surfaces are substantially parallel. The embodiment also includes a shaft having a threaded proximal portion and a substantially conical distal portion, and also a shaft longitudinal axis and a boss extending from the shaft substantially perpendicularly to the shaft axis. The shaft is at least partially within the slot, and the conical distal portion has an external surface at least a part of which engages a part of the internal sloped surfaces of the tip portions. A handle portion may be rotatably connected to the base of the body, and has a threaded hole connected to said threaded portion of the shaft, whereby rotation of the handle portion in a first direction moves the shaft relatively proximally so that the distal portion of the shaft forces the tip portions apart, and rotation of the handle portion in a second direction opposite to the first direction moves the shaft relatively distally and allows the arms to move toward the unstressed relative position. The external surface of the second tip portion may also be a rasping surface, and at least one of the tip portions can include at least one hole extending from the external surface of the tip portion to the internal surface of the tip portion. The boss can be positioned on the shaft so that the boss interferes with the body after the shaft has been moved a predetermined amount in a proximal direction, thereby preventing further proximal movement of the shaft and further movement of the tip portions away from each other. Further, the arms can taper from the base toward the tip portions, and the tip portions may have a width greater than the portion of the arms adjacent them.

Embodiments of methods are also disclosed, including a method of preparing an orthopedic implant site including providing an expandable rasping tool having at least two arms forming a slot and a respective tip portion on each arm and a shaft between the arms with a distal extended portion abutting an interior surface of each tip portion, at least one of the tip portions having an external rasping surface; inserting the tool to an orthopedic implant site; moving the shaft proximally and thereby moving the tip portions apart and into contact with one or more tissue portions; and abrading one or more of the tissue portions by shifting the rasping surface against such portions. The moving step can include distracting the tissue portions, and the providing step can include providing a handle rotatably coupled to the body and threadedly coupled to the shaft, and the moving step includes rotating the handle. The abrading step could occur before or after the moving step. An additional moving step can occur after an abrading step so as to maintain contact with the tissue.

### BRIEF DESCRIPTION OF THE DRAWINGS

FIG. 1 is a side elevational view of an embodiment of an adjustable height rasp.

FIG. 2 is a top plan view of the embodiment shown in FIG. 1.

FIG. 3 is a view of the embodiment shown in FIG. 1, taken along the line 3-3 in FIG. 1 and viewed in the direction of the arrows.

FIG. 4 is a view of the embodiment shown in FIG. 1, taken along the line 4-4 in FIG. 1 and viewed in the direction of the arrows.

FIG. 5 is a side elevational view of part of the embodiment shown in FIG. 1.

FIG. 6 is a cross-sectional view, taken along the lines 6-6 in FIG. 5 and viewed in the direction of the arrows, of the embodiment shown in FIG. 5.

FIG. 7 is a cross-sectional view, taken along the lines 7-7 in FIG. 5 and viewed in the direction of the arrows, of the embodiment shown in FIG. 5.

FIG. 8 is an exploded cross-sectional view of parts of the embodiment shown in FIG. 5.

FIG. 9 is a side elevational view of part of the embodiment shown in FIG. 1.

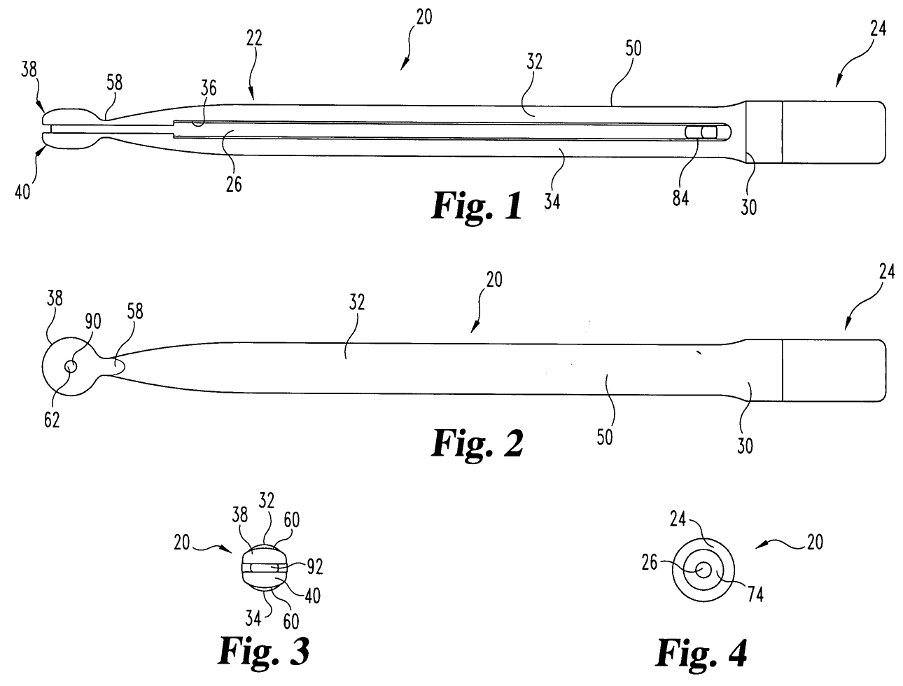

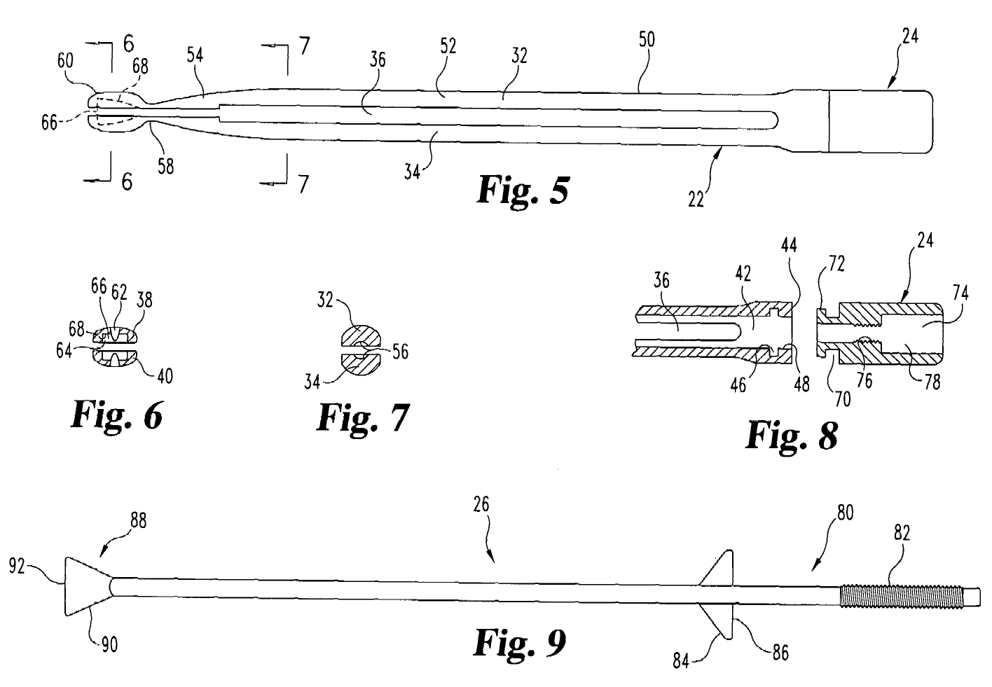

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein being contemplated, as would normally occur to one skilled in the art to which the disclosure relates.

Referring generally to the figures, there is shown an embodiment of a rasp 20 useful in preparing vertebral endplates or other tissues for placement of an implant, as will be further described below. This embodiment of rasp 20 includes main body 22, which is elongated in a particular embodiment, a handle 24, and a central shaft 26. Generally, the surgeon can hold handle 24 and/or a portion of body 22 while placing part of body 22 adjacent to and abrading or otherwise preparing a tissue surface, such as a vertebral endplate. As will be discussed further below, handle 24 can be used to expand at least part of body 22 to provide distraction at the same time.

Body 22, in this embodiment, includes a base portion 30 from which extend two arms 32, 34 separated by a slot 36. Each arm 32, 34 includes a respective tip portion 38, 40. Base portion 30 is substantially cylindrical, at least in part, in this embodiment, and includes a through-hole 42 (FIG. 8) that communicates with slot 36 and an end surface 44. Around hole 42, an internal groove 46 is placed, forming an edge 48 and in a particular embodiment groove 46 lies substantially in a plane that is perpendicular to the central longitudinal axis of hole 42 and the rest of body 22. Base portion 30 tapers from its cylindrical part, in this embodiment, to arms 32, 34.

Arms 32, 34 are substantially identical to each other in this embodiment, and therefore arm 32 will be described in detail for clarity and succinctness. Arm 32 has an external surface 50 that is curved in the illustrated embodiment, and in a particular embodiment forms part of a cylinder (or an arc along surface 50 in a plane perpendicular to the central longitudinal axis of body 22 will form part of a circle). As seen particularly in FIG. 2, arm 32 tapers in width substantially continuously from a location adjacent base 30 to tip portions 38, 40 or a location adjacent them. A middle portion 52 of arm 32 is relatively thin, so that space exists between shaft 26 and arm 32 in slot 36. A distal portion 54 of arm 32 is substantially thicker than middle portion 52, at least in part, and has a hollow or channel 56 that is substantially along the central longitudinal axis of body 22. The channels 56 of the arms 32, 34 together form a conduit for shaft 26, and thus their radii are substantially the same as each other and substantially equal to or slightly larger than a radius of shaft 26, in this embodiment. Distal portion 54 of arm 32 links to tip portion 38, and distal portion 54 of arm 34 links to tip portion 40. In the illustrated embodiment, distal portions 54 narrow to form a neck 58 adjacent tip portions 38, 40. Neck 58 is provided in this embodiment to limit or eliminate abrasion or other contact by parts of rasp 20 with adjacent bone or other tissue. In certain situations, neck 58 may also provide for better visualization of the surgical site, for a place to insert a vacuum tube or other tool to remove abraded material or fluids, or other capabilities.

Tip portions 38, 40 are substantially identical in the illustrated embodiment, and therefore tip portion 38 will be described in detail for clarity and succinctness. Tip portion 38 may be round, and in the illustrated embodiment is substantially circular. An exterior surface 60 is also rounded, and in this embodiment is also roughened (as with knurling, one or more straight edges, or other configuration) to provide a rasping surface. A hole 62 extends through surface 60 to the interior opening or space 64 of tip 38, which can permit abraded material to fall into opening 64 and away from the rasping site. Interior opening 64, in this embodiment, has a distal surface 66 that is relatively flat and a tapering surface 68 that extends substantially from distal surface 66 or a location adjacent to distal surface 66 to channel 56 of arm 32. Since interior opening 64 is significantly larger than channel 56, it will be seen that the taper from distal surface 66 to channel 56 is a narrowing taper.

As noted above, generally tips 38 and 40 are substantially identical to each other, and thus both tips 38 and 40 have an external abrading surface 60 in the illustrated embodiment. In other embodiments, however, one of the external surfaces 60 may lack roughening or other surface features dedicated to rasping or abrading tissue. Thus, one of the external surfaces 60 may be substantially flat, rounded, smooth or otherwise configured to provide a surface to brace against an opposing vertebra or other bone or tissue. As to particular types of rasping surfaces, one or both of surfaces 60 can include one or more protrusions in the form of ridges arranged so that when rasp 20 is moved in a given direction when bodies 30 and 32 are in contact with tissue, the grooves abrade the tissue. Accordingly, if a motion substantially parallel to shaft 26 (i.e. parallel to the central longitudinal axis of rasp 20, in this embodiment) is to be used for abrasion, then the ridges should be substantially perpendicular to shaft 26, or have a directional component that is perpendicular to shaft 26. Similarly, if a motion substantially around the central longitudinal axis is to be used for abrasion, then the ridges should be substantially parallel to shaft 26, or have a directional component that is parallel to shaft 26. Other formations of protrusions or surface configurations that allow the surgeon to scrape endplate material as the rasp is moved across the endplate may be used, including a plurality of pyramid-shaped teeth, frusto-conical projections, spikes, diamond-shaped projections or knurling, or wedge-shaped projections that each extend across the width of the rasping surface, and such protrusions may be formed diagonally to the axis of shaft 26, in chevrons, or otherwise directed.

Handle 24 is substantially cylindrical in the illustrated embodiment, and has an external groove 70 and flange 72 that fit with base portion 30 of body 22. Specifically, flange 72 fits in internal groove 46 of base 30, and the external edge 48 of base 30 fits in groove 70 of handle 24. This is one example of the possible connections between handle 24 and body 22, and others can be used that allow handle 24 to turn with respect to body 22 around the central longitudinal axis of rasp 20. Handle 24 further has an internal opening 74 having a threaded portion 76 and a proximal portion 78, in the illustrated embodiment. Threaded portion 76 may be a relatively medial portion of opening 74, as seen in FIG. 8, or may be in a more distal or more proximal portion of opening 74. Threaded portion 76 mates with a part of shaft 26, as discussed below, and thus may include a standard variety of machine threads. Proximal portion 78 of opening 74 allows an easy visual check of the link between handle 24 and shaft 26, to ensure proper operation, and can also permit lubrication of that link, if necessary. In other embodiments, opening 74 may not have a larger proximal portion 78, or such proximal portion 78 as it may have may not be open to the outside.

Shaft 26 is relatively thin compared to body 22 and handle 24, in this embodiment, and extends through body 22 (within slot 36) and into handle 24. A proximal portion 80 of shaft 26 includes threads 82 compatible with threaded portion 76 of opening 74 of handle 24. In the middle of shaft 26, there is a boss 84, which in the illustrated embodiment is substantially wing-shaped, with a relatively flat blocking surface 86 that extends to either side of shaft 26. In other embodiments, boss 84 (if present) may have other shapes, or may extend to only one side of shaft 26. Boss 84 is placed on shaft 26 so that when shaft 26 is connected to body 22, boss 84 is relatively close to the end of slot 36 and base portion 30. A distal end 88 of shaft 26 is flared, so that it describes a substantially conical external surface 90 with a relatively large diameter end surface 92. Other parts of shaft 26, in the illustrated embodiment, are of a substantially constant diameter. Shaft 26 is a monolithic piece, in the illustrated embodiment, but in other embodiments could be assembled from individual pieces as by welding, gluing, threaded or other mechanical connection.

As previously noted, handle 24 is assembled to body 22 so that handle 24 can be turned or rotated with respect to body 22, and shaft 26 is assembled to body 22 and handle 24 so that shaft 26 is between arms 32, 34 and coupled to handle 24. Thus, in the illustrated embodiment threads 82 of shaft 26 engage the threaded opening 76 of handle 24, so that when handle 24 is turned or rotated, the threads interact so that shaft 26 translates with respect to handle 24 and body 22. In a particular embodiment, where handle 24 is rotated clockwise with respect to body 22 (as viewed from the proximal end of rasp 20), shaft 26 is pulled toward handle 24. Conversely, where handle 24 is rotated counterclockwise, shaft 26 will be pushed toward tips 38, 40. In the illustrated embodiment, shaft 26 either cannot be pushed so that its end 88 passes distal surface 66 in tips 38, 40, or can be so pushed only with difficulty. Further, in the illustrated embodiment shaft 26 can be pulled toward handle 24 only an amount determined by boss 84. When boss 84 engages the end of slot 36 and/or another part of body 22, then shaft 26 cannot be further pulled toward handle 24.

Shaft 26 remains between arms 32, 34 so that a portion of shaft 26 is within channels 56 of arms 32 and 34, and so that end 88 of shaft 26 is within interior spaces 64 of tip portions 38, 40. In one particular embodiment, body 22 is constructed so that without the presence of end 88 of shaft 26 between tips 38 and 40, arms 32, 34 have a rest position in which tips 38 and 40 are either touching or are closer together than they are when end 88 is between them. In other words, the diameter of end surface 92 is at least slightly larger than the distance between tips 38 and 40 in a rest or unstressed state, and when end 88 is between tips 38 and 40, tips 38 and 40 tend to press against end 88. Surface 90 of end 88 is adjacent or facing tapering surfaces 68 within tips 38 and 40. Boss 84 is within slot 36 between arms 32, 34, and relatively near base portion 30 of body 22. In general, as discussed further below, movement of shaft 26 so that its end 88 moves in a proximal direction (e.g. toward handle 24) will spread tip portions 38, 40, as surface 90 pushes against both tip portions. Movement of shaft 26 in the opposite direction, conversely, moves end 88 toward the larger portion of interior spaces 64 of tips 38 and 40, allowing the spring stresses on arms 32 and 34 to move tips 38 and 40 toward each other.

The use of rasp 20 will now be discussed in the context of preparing endplates of adjacent vertebrae for the placement of an intervertebral prosthesis, fusion cage or other type of implant. It will be seen that rasp 20 may be used in a variety of surgical situations, and thus the spinal orthopedic context should not be considered limiting.

Once access has been established to a surgical site proximate to two adjacent vertebrae, the site may be further prepared for the placement of an implant. For example, at least some of the cartilaginous disc material between the vertebrae may be removed in order to open a space for the implant. Other steps may also be performed, such as removal or retraction of other tissues or repair of fractures or other trauma. When the surgeon is ready to place the implant, rasp 20 may be employed. If not already in that state, rasp 20 can be adjusted so that shaft 26 is all the way forward and/or tips 38 and 40 are at their closest, or so that shaft 26 is at a position that causes external surfaces 60 of tips 38 and 40 to be close enough together so as to be easily inserted between the endplates of the adjacent vertebrae. Rasp 20 is maneuvered so that tips 38 and 40 are between the vertebral endplates. Once in that position, the surgeon can turn handle 24 in a first direction (e.g. clockwise) to spread tips 38 and 40 until tips 38 and 40 engage the vertebral endplates. If desired, the surgeon can turn handle 24 further to exert a spreading force on tips 38 and 40 and thus exert distraction force on the vertebral endplates to force them further apart. The surgeon can then work rasp 20 by translational motion substantially along the central longitudinal axis of rasp 20, by rotation substantially around that axis, and/or by rotation in a plane parallel or near parallel to the plane of the intervertebral space. Such motion of rasp 20 when tips 38 and 40 engage the adjacent endplates cause abrading of the endplates by roughened exterior surfaces 60 of tips 38 and 40. As the endplates are abraded, fluids or material can be moved away from the abrading site through holes 62 in tips 38, 40, or compositions can be introduced to the site via holes 62. Further, as a hollow is created by such abrading, the surgeon can again (or periodically) turn handle 24 to spread tips 38 and 40 further apart and into pressing contact with the endplates.

Using rasp 20, the surgeon can create a hollow or opening suitable for placement of an implant, of osteogenic material (e.g. autograft, allograft, BMP, LIM mineralization protein, or other substances) or medicaments, or of a combination or other items. In the case of a substantially cylindrical fusion cage, for example, the surgeon can expand rasp 20 within an intervertebral space so that the distance between exterior surfaces 60 of tips 38 and 40 is approximately the diameter of the cage, and can work rasp 20 to create substantially part-cylindrical hollows in both endplates. Such part-cylindrical hollows will have a diameter that is approximately that of the cage, and therefore the cage will fit closely in the hollows in the endplates. With a more rectangular spacer implant, rasp 20 can similarly be expanded and worked to create appropriate hollows of a shape and depth to accommodate the implant. Rasp 20 can also be used to even out endplate surfaces rather than creating hollows, for instance if intervertebral spacers or prostheses with broad, flat endplate-engaging surfaces are being implanted.

Once the desired hollows or other rasping task has been accomplished, rasp 20 can be removed from between the adjacent vertebrae. In many cases it will be desirable to turn handle 24 in a second direction that may be opposite to the first direction that expands tips 38 and 40 (e.g. counterclockwise) to allow tips 38 and 40 to move together prior to removing rasp 20 as arms 32, 34 attempt to resume an unstressed relative position. In such cases, the surgeon can insert a temporary spacer or tool to maintain the desired space between the vertebrae, if that space will not be naturally maintained by the vertebrae. In other cases, rasp 20 may be directly removed from between the vertebrae without reducing the space between tips 38 and 40. The surgeon can then insert the implant, substance or combination he or she desires in between the vertebrae, and the procedure can be concluded.

Rasp 20 may be made of a variety of sturdy biocompatible surgical-grade materials, including metals such as titanium and stainless steel. Embodiments of rasp 20 may be made for multiple uses, either during the same operation on a given patient, or for multiple patients following suitable sterilization procedures. As noted above, maintenance (such as lubrication) of multiple-use embodiments of rasp 20 can be performed between uses.

While the disclosure has been illustrated and described in detail in the drawings and foregoing description, the same is to be considered as illustrative and not restrictive in character, it being understood that all changes and modifications that come within the spirit of the disclosure are desired to be protected. Directional terms used herein, such as “above,” “below” or similar terms, are not intended to be necessarily indicative of the orientation of the described feature in all situations. Rather, they are used in conjunction with the figures to indicate general relationships between or among features.

What is claimed is:

1. A tool for use in spinal orthopedic surgery, comprising:
a handle having a longitudinal axis;
a body having a base connected to said handle and a plurality of arms each having a tip portion, each said tip portion having a relatively internal sloped surface and a relatively external surface, at least part of at least one of said external surfaces being a rasping surface, and said arms have an unstressed position relative to each other; and
a shaft having a proximal portion operatively connected to said handle and a distal portion, said distal portion having an expanded part that contacts said relatively internal sloped portions of said tip portions, whereby operating said handle in a first mode moves said expanded part against said relatively internal sloped portions to force said arms apart, and operating said handle in a second mode moves said expanded part relative to said relatively internal sloped portions to allow said arms to move toward said unstressed relative position.

2. The apparatus of claim 1, wherein said handle is rotatable with respect to said body and is threadedly connected to said shaft, and wherein said first mode includes turning said handle relative to said body in a first direction, and said second mode includes turning said handle relative to said body in a second direction that is substantially opposite to said first direction.

3. The apparatus of claim 1, wherein said body includes only two arms, and in said unstressed relative position of said arms, said arms define a slot between them.

4. The apparatus of claim 3, wherein said shaft includes a boss that extends through at least part of said slot.

5. The apparatus of claim 4, wherein said shaft includes a longitudinal axis, and said boss extend to either side of said shaft and substantially perpendicularly to said axis.

6. The apparatus of claim 3, wherein said shaft extends within said slot and through said base.

7. The apparatus of claim 1, wherein said extended part of said shaft is tapered.

8. The apparatus of claim 7, wherein said extended part of said shaft forms at least part of a cone.

9. The apparatus of claim 1, wherein at least one of said tip portions include at least one hole from said relatively exterior surface to said relatively interior surface.

10. The apparatus of claim 9, wherein said shaft has a longitudinal axis and said at least one hole is substantially perpendicular to said axis.

11. The apparatus of claim 1, wherein said shaft has a longitudinal axis and wherein at least one of said tip portions includes a relatively distal surface adjacent said relatively internal surface, said distal surface being substantially perpendicular to said longitudinal axis.

12. The apparatus of claim 1, wherein said arms taper from said base toward said tip portions, and said tip portions have a width greater than the portion of the arms adjacent them.

13. An orthopedic surgical apparatus comprising:
a body having a base, first and second longitudinal arms extending from said defining a slot between them, and a longitudinal axis along said slot, said base having a hole therethrough substantially along said axis, said first arm having an internal surface and a first tip portion, said first tip portion having an external rasping surface and an internal sloped surface, said second arm having an internal surface facing said internal surface of said first arm and a second tip portion facing said first tip portion, said second tip portion having an external surface and an internal sloped surface, and wherein said first and second arms have an unstressed relative position such that said internal surfaces are substantially parallel;
a shaft having a threaded proximal portion and a substantially conical distal portion, said shaft having a shaft longitudinal axis and a boss extending from said shaft substantially perpendicularly to said shaft axis, said shaft being at least partially within said slot, and said conical distal portion having an external surface at least a part of which engages a part of said internal sloped surfaces of said tip portions; and
a handle portion rotatably connected to said base of said body and having a threaded hole connected to said threaded portion of said shaft, whereby rotation of said handle portion in a first direction moves said shaft relatively proximally so that said distal portion of said shaft forces said tip portions apart, and rotation of said handle portion in a second direction opposite to said first direction moves said shaft relatively distally and allows said arms to move toward said unstressed relative position.

14. The apparatus of claim 13, wherein said external surface of said second tip portion is a rasping surface.

15. The apparatus of claim 13, wherein at least one of said tip portions includes at least one hole extending from said external surface of said tip portion to said internal surface of said tip portion.

16. The apparatus of claim 13, wherein said boss is positioned on said shaft so that said boss interferes with said body after said shaft has been moved a predetermined amount in a proximal direction, thereby preventing further proximal movement of said shaft and further movement of said tip portions away from each other.

17. The apparatus of claim 13, wherein said arms taper from said base toward said tip portions, and said tip portions have a width greater than the portion of the arms adjacent them.

18. A method of preparing an orthopedic implant site, comprising:
providing an expandable rasping tool having at least two arms forming a slot and a respective tip portion on each arm and a shaft between said arms with a distal extended portion abutting an interior surface of each said tip portion, at least one of said tip portions having an external rasping surface;
inserting said tool to an orthopedic implant site;
moving said shaft proximally and thereby moving said tip portions apart and into contact with one or more tissue portions; and
abrading one or more of said tissue portions by shifting said rasping surface against such portions.

19. The method of claim 17 wherein said moving step includes distracting said tissue portions.

20. The method of claim 17, wherein said providing step includes providing a handle rotatably coupled to said body and threadedly coupled to said shaft, and said moving step includes rotating said handle.

21. The method of claim 17, wherein said abrading step occurs after said moving step.

22. The method of claim 21, further comprising a second moving said shaft proximally and thereby moving said tip portions apart to maintain contact with said tissue portions after said abrading step.
